(19) United States
(12) Patent Application Publication
(10) Pub. No.: US 2010/0298663 A1
(43) Pub. Date: Nov. 25, 2010

(54) SYSTEM AND METHOD FOR DETECTION AND TREATMENT OF IRREGULAR METABOLIC FUNCTION

(75) Inventors: Xiangqun Chen, Valencia, CA (US); Keith Victorine, Sherman Oaks, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/469,582

(22) Filed: May 20, 2009

(51) Int. Cl. A61B 5/00

(52) U.S. Cl. 600/301; 600/300

(57) ABSTRACT

A system and method for detecting and treating irregular metabolic function. The systems and methods generally include an implantable medical device that measures various physiological parameters. Such physiological parameters generally provide an indication of the patient's metabolic rate, respiratory rate, and activity level. For example, the medical device may measure the patient's relative movement, or may measure the patient's oxygen consumption. These measures are then recorded, and the recorded data is used to determine the normal relationship between metabolic rate and other physiological parameters for that patient. The baseline relationship is then used to determine if the patient experiences an increase or decrease in metabolic rate that is not explained by changes in other physiological parameters. Further testing, and treatment if necessary, can be performed if the physiological parameters indicate irregular metabolic function.

### FIELD OF THE INVENTION

The present invention relates generally to implantable medical devices. More particularly, the invention relates to systems and methods for detecting the presence of metabolic related diseases or other illnesses which cause deviation from a patient's expected metabolic rate.

### BACKGROUND

Metabolic diseases affect a large number of men and women. The prevalence of these diseases, particularly hypothyroidism and hyperthyroidism, increases as the patient ages. Medicines taken for other problems can also interfere with normal metabolic function in some patients.

Hypothyroidism, for example, is a condition in which the thyroid releases insufficient thyroid hormone, resulting in symptoms associated with a slow metabolism. These symptoms can include fatigue, weakness, weight gain, muscle cramps and aches, depression, and memory loss, among others. Hypothyroidism is one of the most common endocrine disorders encountered in clinical practice. Studies suggest that as many as 10 percent of women have some degree of thyroid hormone deficiency. Up to 20 percent of women older than 60 have some degree of hypothyroidism.

Hyperthyroidism is a condition in which the thyroid releases too much thyroid hormone, resulting in symptoms associated with an abnormally high metabolism. These symptoms can include palpitations, heat intolerance, nervousness, insomnia, breathlessness, light or absent menstrual periods, fast heart rate, and weight loss, among others. Symptoms of hyperthyroidism can progress gradually, with the effect that an effected patient may not realize there is a problem until the symptoms become more severe. Hyperthyroidism is less prevalent than hypothyroidism, with studies estimating that approximately 2 percent of women and 0.2 percent of men have some degree of the condition. Both hypothyroidism and hyperthyroidism respond well to treatment, especially if detected at early onset.

Metabolic function can vary depending on factors such as a patient's activity level or thyroid hormone production. What is needed, then, is a method for early detection of abnormal metabolic function. Thus, information regarding the patient's current metabolic rate, current activity level, and historical data relating to the patients metabolic rate at various activity levels would help identify possible abnormal metabolic function.

### BRIEF SUMMARY OF THE INVENTION

Presented herein are various systems and methods for detecting the presence of metabolic related diseases or other irregularities which cause deviation from a patient's expected metabolic rate. The systems and methods generally include an implantable medical device that measures various physiological parameters. Such physiological parameters generally provide an indication of, for example, the patient's metabolic rate, activity level, and respiratory rate. For example, the medical device may measure the patient's relative movement, or may measure the patient's oxygen consumption. The medical device may also track trends in the patient's metabolic rate as compared to other physiological parameters. These measures are then used to determine whether, based on the patient's current activity level or respiratory rate, the patient's metabolic rate is abnormally high or low. Once a predetermined threshold variance is exceeded, the implantable device may trigger a notification signal to the patient and/or the patient's physician.

Further embodiments, features, and advantages of the presented systems and methods, as well as the structure and operation of the various embodiments of the present systems and methods, are described in detail below with reference to the accompanying drawings.

### BRIEF DESCRIPTION OF THE DRAWINGS/FIGURES

The accompanying drawings, which are incorporated herein and form part of the specification, illustrate the methods and systems presented herein for detecting and treating metabolic related diseases or other irregularities. Together with the detailed description, the drawings further serve to explain the principles of and to enable a person skilled in the relevant art(s) to make and use the methods and systems presented herein.

In the drawings, like reference numbers indicate identical or functionally similar elements. Further, the drawing in which an element first appears is typically indicated by the leftmost digit(s) in the corresponding reference number (e.g., an element numbered 302 first appears in FIG. 3).

FIG. 1 is a simplified diagram illustrating an exemplary implantable cardiac device (ICD) for use in the methods presented herein.

FIG. 2 is a functional block diagram of an exemplary ICD.

FIG. 3 is a functional block diagram of the internal architecture and principle external connections of an exemplary external programming device which may be used by a human programmer to monitor or program an ICD.

FIG. 4 is a high-level block diagram of a method of detecting irregularities in metabolic function in a patient.

FIG. 5 is a flow chart showing a method in accordance with one embodiment described herein.

FIG. 6 is a flow chart showing a method in accordance with another embodiment described herein.

### DETAILED DESCRIPTION OF THE INVENTION

### 1. Overview

The following detailed description of methods and systems for detecting and treating irregular metabolic function refers to the accompanying drawings that illustrate exemplary embodiments consistent with these methods and systems. Other embodiments are possible, and modifications may be made to the embodiments within the spirit and scope of the methods and systems presented herein. Therefore, the following detailed description is not meant to limit the methods and systems described herein. Rather, the scope of these methods and systems is defined by the appended claims.

It would be apparent to one of skill in the art that the methods and systems for detection and treatment of irregular metabolic function, as described below, may be implemented in many different embodiments of hardware, software, firmware, and/or the entities illustrated in the figures. Any actual software and/or hardware described herein is not limiting of these methods and systems. Thus, the operation and behavior of the methods and systems will be described with the understanding that modifications and variations of the embodiments are possible, given the level of detail presented herein.

As used herein, the term “patient” is intended to broadly include any individual who may benefit from the systems and methods described herein. As such, the term “patient” should not be limited to individuals subject to the traditional patient-physician relationship. The term “patient” should therefore also include individuals who are clients of any persons or entities selling, using, performing, and/or directing the performance of the disclosed systems and/or methods. The terms “physician” and “healthcare service provider” are used interchangeably and are intended to broadly include any individuals or entities who sell, use, perform, and/or direct the performance of the disclosed systems and/or methods. For example, a “healthcare service provider” may include, but is not limited to, a doctor, a nurse, a dietician, a caretaker, a pharmacist, or a business entity. In some instances, the “healthcare service provider” may be the actual “patient.” For example, if an individual is performing the disclosed methods for his own benefit, then the individual is both the “patient” and the “healthcare service provider.” The term “manage” (or any derivative of this term) is intended to mean “to direct,” or “to take charge of,” or “to attempt to control or effectuate.” The phrase “irregular metabolic function” (or any derivative of this phrase) is a broad phrase which includes any condition in which the patient's metabolic system does not perform as expected for a particular patient. Irregular metabolic function can be caused by a variety of factors, including, but not limited to, thyroid disease, medicinal side-effects, etc.

### 2. Exemplary Environment—Overview

Before describing in detail the methods and systems for detection and treatment of irregular metabolic function, it is helpful to describe an example environment in which these methods and systems may be implemented. The methods and systems described herein may be particularly useful in the environment of an implantable cardiac device (ICD) which is programmed via an external general purpose computer or via an external dedicated ICD programming device.

An ICD is generally a physiologic measuring device and therapeutic device that is implanted in a patient to monitor cardiac function and to deliver appropriate electrical therapy, for example, pacing pulses, cardioverting and defibrillator pulses, and drug therapy, as required. ICDs include, for example and without limitation, pacemakers, cardioverters, defibrillators, implantable cardioverter defibrillators, implantable cardiac rhythm management devices, and the like. Such devices may also be used in particular to monitor cardiac electrical activity and to analyze cardiac electrical activity. The term “implantable cardiac device” or simply “ICD” is used herein to refer to any such implantable cardiac device.

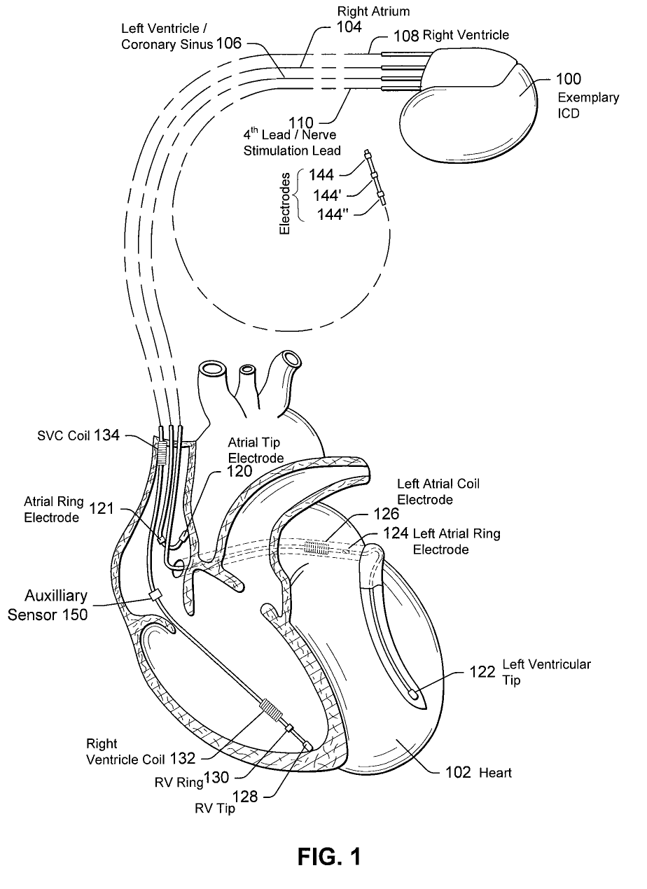

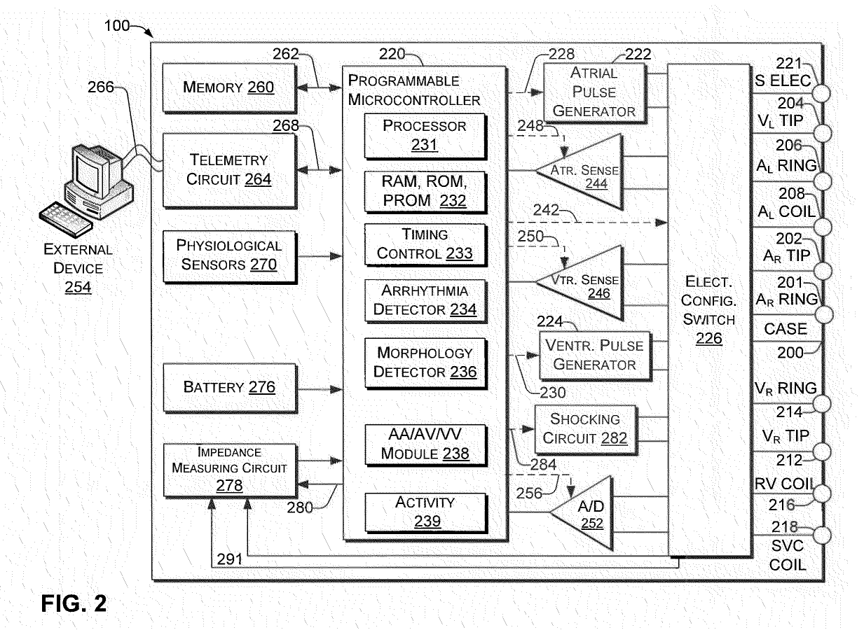

FIGS. 1 and 2 illustrate such an environment.

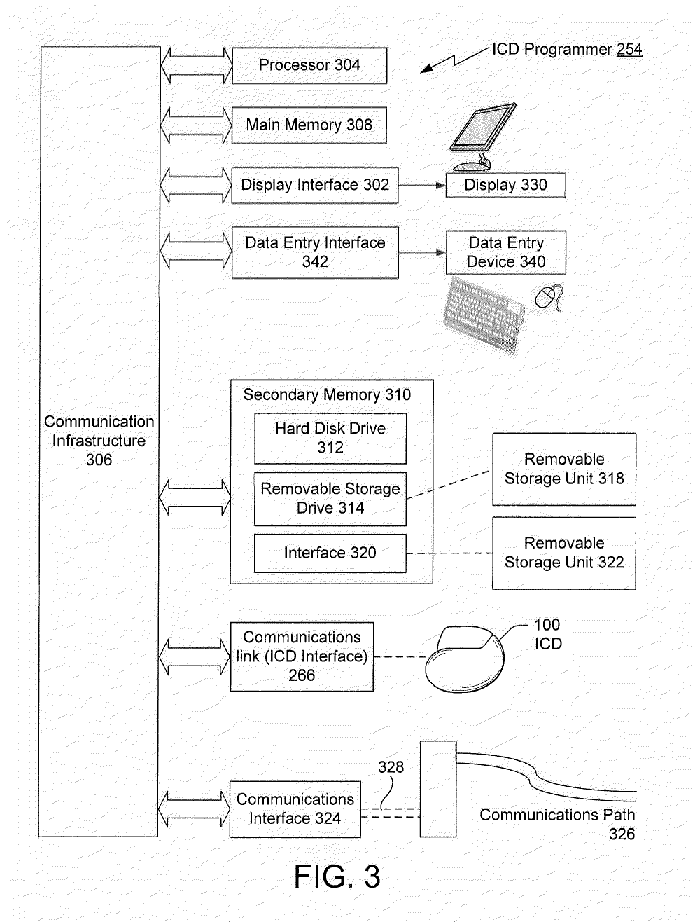

FIG. 3 illustrates the architecture of an external programming device which may be used by a human programmer to monitor, program, or interact with an ICD.

### 3. Exemplary ICD in Electrical Communication With a Patient's Heart

The techniques described below are intended to be implemented in connection with any ICD or any similar device that is configured or configurable to measure selective physiological parameters indicative of a patient's metabolic function.

FIG. 1 shows an exemplary ICD 100 in electrical communication with a patient's heart 102 by way of three leads 104, 106, 108, suitable for sensing cardiac rhythms and delivering multi-chamber stimulation and/or shock therapy. The leads 104, 106, 108 are optionally configurable for delivery of stimulation pulses suitable for stimulation of autonomic nerves. In addition, the ICD 100 includes a fourth lead 110 having, in this implementation, three electrodes 144, 144′, 144″ suitable for stimulation of autonomic nerves. Lead 110 may be positioned in and/or near a patient's heart or near an autonomic nerve within a patient's body and remote from the heart. Of course, such a lead may be positioned epicardially or at some other location to stimulate other tissue.

The right atrial lead 104, as the name implies, is positioned in and/or passes through a patient's right atrium. The right atrial lead 104 optionally senses atrial cardiac signals and/or provides right atrial chamber stimulation therapy. As shown in FIG. 1, ICD 100 is coupled to an implantable right atrial lead 104 having, for example, an atrial tip electrode 120, which typically is implanted in the patient's right atrial appendage. The lead 104, as shown in FIG. 1, also includes an atrial ring electrode 121. Of course, the lead 104 may have other electrodes as well. For example, the right atrial lead optionally includes a distal bifurcation having electrodes suitable for stimulation of autonomic nerves.

To sense atrial cardiac signals, ventricular cardiac signals and/or to provide chamber pacing therapy, particularly on the left side of a patient's heart, ICD 100 is coupled to a coronary sinus lead 106 designed for placement in the coronary sinus and/or tributary veins of the coronary sinus. Thus, the coronary sinus lead 106 is optionally suitable for positioning at least one distal electrode adjacent to the left ventricle and/or additional electrode(s) adjacent to the left atrium. In a normal heart, tributary veins of the coronary sinus include, but may not be limited to, the great cardiac vein, the left marginal vein, the left posterior ventricular vein, the middle cardiac vein, and the small cardiac vein.

Accordingly, an exemplary coronary sinus lead 106 is optionally designed to receive atrial and ventricular cardiac signals and to deliver left ventricular pacing therapy using, for example, at least a left ventricular tip electrode 122, left atrial pacing therapy using at least a left atrial ring electrode 124, and shocking therapy using at least a left atrial coil electrode 126. For a complete description of a coronary sinus lead, the reader is directed to U.S. Pat. No. 5,466,254, “Coronary Sinus Lead with Atrial Sensing Capability” (Helland), which is incorporated herein by reference in its entirety. The coronary sinus lead 106 further optionally includes electrodes for stimulation of autonomic nerves. Such a lead may include pacing and autonomic nerve stimulation functionality and may further include bifurcations or legs. For example, an exemplary coronary sinus lead includes pacing electrodes capable of delivering pacing pulses to a patient's left ventricle and at least one electrode capable of stimulating an autonomic nerve. An exemplary coronary sinus lead (or left ventricular lead or left atrial lead) may also include at least one electrode capable of stimulating an autonomic nerve, such an electrode may be positioned on the lead or a bifurcation or leg of the lead.

ICD 100 is also shown in electrical communication with the patient's heart 102 by way of an implantable right ventricular (RV) lead 108 having, in this exemplary implementation, a right ventricular tip electrode 128, a right ventricular ring electrode 130, a right ventricular coil electrode 132, a superior vena cava (SVC) coil electrode 134, and an auxiliary sensor 150. Typically, the right ventricular lead 108 is transvenously inserted into the heart 102 to place the right ventricular tip electrode 128 in the right ventricular apex so that the RV coil electrode 132 will be positioned in the right ventricle and the SVC coil electrode 134 will be positioned in the superior vena cava. Accordingly, RV lead 108 is capable of sensing or receiving cardiac signals, and delivering stimulation in the form of pacing and shock therapy to the right ventricle. RV lead 108 is also capable of receiving physiological data from the RV through auxiliary sensor 150. While auxiliary sensor 150 is shown in the RV, one of skill in the art would understand that auxiliary sensor 150 may be positioned anywhere that is appropriate for the intended use of the sensor. For example, auxiliary sensor 150 may be positioned in the right atrium of the heart, or in the pulmonary arteries. If auxiliary sensor 150 is an oxygen sensor, then RV lead 108 and auxiliary sensor 150 may be used to measure the oxygen concentration of the blood entering the RV. Alternatively, auxiliary sensor 150 may be used alone or in conjunction with other components of ICD 100 to measure any physiological parameter of the patient's; for example, the patient's temperature, lateral movement, arterial oxygen saturation (SaO₂), venous oxygen saturation (SvO₂), cardiac output, respiratory rate, respiratory volume, minute ventilation, and/or blood pressure. An exemplary RV lead may also include at least one electrode capable of stimulating an autonomic nerve, such an electrode may be positioned on the lead or a bifurcation or leg of the lead.

### 4. Functional Elements of an Exemplary ICD

FIG. 2 shows an exemplary, simplified block diagram depicting various components of ICD 100. ICD 100 can be capable of monitoring and/or treating for irregular metabolic function, treating both fast and slow arrhythmias with stimulation therapy, including cardioversion, defibrillation, and pacing stimulation, or delivering stimuli to autonomic nerves. While a particular multi-chamber device is shown, it is to be appreciated and understood that this is done for illustration purposes only. Thus, the techniques and methods described below can be implemented in connection with any suitably configured or configurable implantable medical device. Accordingly, one of skill in the art could readily duplicate, eliminate, or disable the appropriate circuitry in any desired combination to provide a device capable of monitoring and/or treating for abnormal metabolic function.

Housing 200 for ICD 100 is often referred to as the “can”, “case” or “case electrode”, and may be programmably selected to act as the return electrode for all “unipolar” modes. Housing 200 may further be used as a return electrode alone or in combination with one or more of the coil electrodes 126, 132 and 134 (see FIG. 1) for shocking purposes. Housing 200 further includes a connector (not shown) having a plurality of terminals 201, 202, 204, 206, 208, 212, 214, 216, 218, 221 (shown schematically and, for convenience, the names of the electrodes to which they are connected are shown next to the terminals).

To achieve right atrial sensing, pacing and/or autonomic stimulation, the connector includes at least a right atrial tip terminal (AR TIP) 202 adapted for connection to the atrial tip electrode 120. A right atrial ring terminal (AR RING) 201 is also shown, which is adapted for connection to the atrial ring electrode 121. To achieve left chamber sensing, pacing, shocking, and/or autonomic stimulation, the connector includes at least a left ventricular tip terminal (VL TIP) 204, a left atrial ring terminal (AL RING) 206, and a left atrial shocking terminal (AL COIL) 208, which are adapted for connection to the left ventricular tip electrode 122, the left atrial ring electrode 124, and the left atrial coil electrode 126, respectively. Connection to suitable autonomic nerve stimulation electrodes is also possible via these and/or other terminals (e.g., via a nerve stimulation terminal S ELEC 221).

To support right chamber sensing, pacing, shocking, and/or autonomic nerve stimulation, the connector further includes a right ventricular tip terminal (VR TIP) 212, a right ventricular ring terminal (VR RING) 214, a right ventricular shocking terminal (RV COIL) 216, and a superior vena cava shocking terminal (SVC COIL) 218, which are adapted for connection to the right ventricular tip electrode 128, right ventricular ring electrode 130, the RV coil electrode 132, and the SVC coil electrode 134, respectively. Connection to suitable autonomic nerve stimulation electrodes is also possible via these and/or other terminals (e.g., via the nerve stimulation terminal S ELEC 221).

At the core of ICD 100 is a programmable microcontroller 220. As is well known in the art, microcontroller 220 typically includes a processor or microprocessor 231, or equivalent control circuitry, designed specifically for controlling the delivery of stimulation therapy, and may further include onboard memory 232 (which may be, for example and without limitation, RAM, ROM, PROM, one or more internal registers, etc.), logic and timing circuitry, state machine circuitry, and I/O circuitry. Typically, microcontroller 220 includes the ability to process or monitor input signals (data or information) as controlled by a program code stored in a designated block of memory. The type of microcontroller is not critical to the described implementations. Rather, any suitable microcontroller 220 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. No. 4,712,555 (Thornander) and U.S. Pat. No. 4,944,298 (Sholder), all of which are incorporated by reference herein in its entirety. For a more detailed description of the various timing intervals used within the stimulation device and their inter-relationship, see U.S. Pat. No. 4,788,980 (Mann et al.), also incorporated herein by reference.

FIG. 2 also shows an atrial pulse generator 222 and a ventricular pulse generator 224 that generate pacing stimulation pulses for delivery by the right atrial lead 104, the coronary sinus lead 106, and/or the right ventricular lead 108 via an electrode configuration switch 226. It is understood that in order to provide stimulation therapy in each of the four chambers of the heart (or to autonomic nerves or other tissue) the atrial and ventricular pulse generators, 222 and 224, may include dedicated, independent pulse generators, multiplexed pulse generators, or shared pulse generators. The pulse generators 222 and 224 are controlled by the microcontroller 220 via appropriate control signals 228 and 230, respectively, to trigger or inhibit the stimulation pulses.

Microcontroller 220 further includes timing control circuitry 233 to control the timing of the stimulation pulses (e.g., pacing rate, atrio-ventricular (e.g., AV) delay, atrial interconduction (AA) delay, or ventricular interconduction (VV) delay, etc.) as well as to keep track of the timing of refractory periods, blanking intervals, noise detection windows, evoked response windows, alert intervals, marker channel timing, etc., which is well known in the art.

Microcontroller 220 further includes an arrhythmia detector 234, a morphology detector 236, and optionally an orthostatic compensator and a minute ventilation (MV) response module (the latter two are not shown in FIG. 2). These components can be utilized by ICD 100 for determining desirable times to administer various therapies. The aforementioned components may be implemented in hardware as part of the microcontroller 220, or as software/firmware instructions programmed into the device and executed on the microcontroller 220 during certain modes of operation.

Microcontroller 220 further includes an AA delay, AV delay and/or VV delay module 238 for performing a variety of tasks related to AA delay, AV delay and/or VV delay. This component can be utilized by ICD 100 for determining desirable times to administer various therapies, including, but not limited to, ventricular stimulation therapy, bi-ventricular stimulation therapy, resynchronization therapy, atrial stimulation therapy, etc. The AA/AV/VV module 238 may be implemented in hardware as part of the microcontroller 220, or as software/firmware instructions programmed into the device and executed on the microcontroller 220 during certain modes of operation. Of course, such a module may be limited to one or more of the particular functions of AA delay, AV delay and/or VV delay. Such a module may include other capabilities related to other functions that may be germane to the delays. Such a module may help make determinations as to fusion.

The microcontroller 220 of FIG. 2 also includes an activity module 239. This module may include control logic for one or more activity related features. For example, the module 239 may include an algorithm for determining patient activity level, calling for an activity test, calling for a change in one or more pacing parameters, etc. The module 239 may be implemented in hardware as part of the microcontroller 220, or as software/firmware instructions programmed into the device and executed on the microcontroller 220 during certain modes of operation. The module 239 may act cooperatively with the AA/AV/VV module 238.

The electrode configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, combipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, and the right ventricular lead 108, through the switch 226 for detecting the presence of cardiac activity in each of the four chambers of the heart. Accordingly, the atrial (ATR. SENSE) and ventricular (VTR. SENSE) sensing circuits, 244 and 246, may include dedicated sense amplifiers, multiplexed amplifiers, or shared amplifiers. Switch 226 determines the “sensing polarity” of the cardiac signal by selectively closing the appropriate switches, as is also known in the art. In this way, the clinician may program the sensing polarity independent of the stimulation polarity. The sensing circuits (e.g., 244 and 246) are optionally capable of obtaining information indicative of tissue capture.

Each sensing circuit 244 and 246 preferably employs one or more low power, precision amplifiers with programmable gain and/or automatic gain control, bandpass filtering, and a threshold detection circuit, as known in the art, to selectively sense the cardiac signal of interest. The automatic gain control enables ICD 100 to deal effectively with the difficult problem of sensing the low amplitude signal characteristics of atrial or ventricular fibrillation.

The outputs of the atrial and ventricular sensing circuits 244 and 246 are connected to the microcontroller 220, which, in turn, is able to trigger or inhibit the atrial and ventricular pulse generators 222 and 224, respectively, in a demand fashion in response to the absence or presence of cardiac activity in the appropriate chambers of the heart. Furthermore, as described herein, the microcontroller 220 is also capable of analyzing information output from the sensing circuits 244 and 246 and/or the analog-to-digital (A/D) data acquisition system 252 to determine or detect whether and to what degree tissue capture has occurred and to program a pulse, or pulses, in response to such determinations. The sensing circuits 244 and 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits, 244 and 246, as is known in the art.

For arrhythmia detection, ICD 100 utilizes the atrial and ventricular sensing circuits, 244 and 246, to sense cardiac signals to determine whether a rhythm is physiologic or pathologic. In reference to arrhythmias, as used herein, “sensing” is reserved for the noting of an electrical signal or obtaining data (information), and “detection” is the processing (analysis) of these sensed signals and noting the presence of an arrhythmia. In some instances, detection or detecting includes sensing and in some instances sensing of a particular signal alone is sufficient for detection (e.g., presence/absence, etc.).

The timing intervals between sensed events (e.g., P-waves, R-waves, and depolarization signals associated with fibrillation which are sometimes referred to as “F-waves” or “Fib-waves”) are then classified by the arrhythmia detector 234 of the microcontroller 220 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Cardiac signals are also applied to inputs of an analog-to-digital (A/D) data acquisition system 252. The data acquisition system 252 is configured to acquire intracardiac electrogram (EGM) signals, convert the raw analog data into a digital signal, and store the digital signals for later processing and/or telemetric transmission to an external device 254. Data acquisition system 252 may be configured by microcontroller 220 via control signals 256. The data acquisition system 252 is coupled to the right atrial lead 104, the coronary sinus lead 106, the right ventricular lead 108 and/or the nerve stimulation lead 110 through the switch 226 to sample cardiac signals across any pair of desired electrodes.

The microcontroller 220 is further coupled to a memory 260 by a suitable data/address bus 262, wherein the programmable operating parameters used by the microcontroller 220 are stored and modified, as required, in order to customize the operation of ICD 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. One feature may be the ability to sense and store a relatively large amount of data (e.g., from the data acquisition system 252), which data may then be used for subsequent analysis to guide the programming of the device.

Essentially, the operation of the ICD control circuitry, including but not limited to pulse generators, timing control circuitry, delay modules, the activity module, and sensing and detection circuits, may be controlled, partly controlled, or fine-tuned by a variety of parameters, such as those indicated above which may be stored and modified, and may be set via an external ICD programming device 254.

Advantageously, the operating parameters of ICD 100 may be non-invasively programmed into the memory 260 through a telemetry circuit 264 in telemetric communication via communication link 266 with the external ICD programming device 254, which may be a general purpose computer, a dedicated ICD programmer, a transtelephonic transceiver, or a diagnostic system analyzer. The microcontroller 220 activates the telemetry circuit 264 with a control signal 268. The telemetry circuit 264 advantageously allows intracardiac electrograms and status information relating to the operation of the device 100 (as contained in the microcontroller 220 or memory 260) to be sent to the external device 254 through an established communication link 266. The ICD 100 may also receive human programmer instructions via the external device 254.

ICD 100 further includes a physiological sensor 270. The physiological sensor 270 may be used to detect changes in cardiac output (see, e.g., U.S. Pat. No. 6,314,323, entitled “Heart stimulator determining cardiac output, by measuring the systolic pressure, for controlling the stimulation”, to Ekwall, issued Nov. 6, 2001, which is incorporated herein by reference in its entirety, which discusses a pressure sensor adapted to sense pressure in a right ventricle and to generate an electrical pressure signal corresponding to the sensed pressure, an integrator supplied with the pressure signal which integrates the pressure signal between a start time and a stop time to produce an integration result that corresponds to cardiac output), changes in the physiological condition of the heart, or diurnal changes in activity (e.g., detecting sleep and wake states). Physiological sensor 270 may further include sensors to determine the patient's activity level activity level and/or the patients respiratory rate.

While shown as being included within ICD 100, it is to be understood that the physiological sensor 270 may also be external to ICD 100, yet still be implanted within or carried by the patient. Examples of physiologic sensors that may be implemented in ICD 100 include known sensors that, for example, sense respiration rate, activity level, pH of blood, ventricular gradient, cardiac output, preload, afterload, contractility, hemodynamics, pressure, and so forth. Another sensor that may be used is one that detects activity variance, wherein an activity sensor is monitored diurnally to detect the low variance in the measurement corresponding to the sleep state. For a complete description of an example activity variance sensor, the reader is directed to U.S. Pat. No. 5,476,483 (Bornzin et al.), issued Dec. 19, 1995, which patent is hereby incorporated by reference in its entirety.

More specifically, physiological sensors 270 optionally include sensors for detecting movement and minute ventilation in the patient. Physiological sensors 270 may include a position sensor and/or a minute ventilation (MV) sensor to sense minute ventilation, which is defined as the total volume of air that moves in and out of a patient's lungs in a minute. Signals generated by the position sensor and MV sensor are passed to the microcontroller 220 for analysis in determining whether to adjust the pacing rate, etc. The microcontroller 220 monitors the signals for indications of the patient's position and activity status, such as whether the patient is climbing upstairs or descending downstairs or whether the patient is sitting up or lying down.

ICD 100 additionally includes a battery 276 that provides operating power to all of the circuits shown in FIG. 2. For ICD 100, battery 276 is capable of operating at low current drains for long periods of time (e.g., preferably less than 10 μAmps), and is capable of providing high-current pulses (for capacitor charging) when the patient requires a shock pulse (e.g., preferably, in excess of 2 Amps, at voltages above 2 Volts, for periods of 10 seconds or more). The battery 276 also desirably has a predictable discharge characteristic so that elective replacement time can be detected.

ICD 100 can further include magnet detection circuitry (not shown), coupled to the microcontroller 220, to detect when a magnet is placed over ICD 100. A magnet may be used by a clinician to perform various test functions of ICD 100 and/or to signal the microcontroller 220 that the external programmer 254 is in place to receive or transmit data to the microcontroller 220 through the telemetry circuit 264.

ICD 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 278 is advantageously coupled to the switch 226 via circuit line(s) 291 so that any desired electrode may be used.

In the case where ICD 100 is intended to operate as an implantable cardioverter/defibrillator device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (e.g., up to approximately 0.5 J), moderate (e.g., approximately 0.5 J to approximately 10 J), or high energy (e.g., approximately 11 J to approximately 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV coil electrode 132, or as part of a split electrical vector using the SVC coil electrode 134 or the left atrial coil electrode 126 (i.e., using the RV electrode as a common electrode). Other exemplary devices may include one or more other coil electrodes or suitable shock electrodes (e.g., a LV coil, etc.).

Cardioversion level shocks are generally considered to be of low to moderate energy level (where possible, so as to minimize pain felt by the patient), and/or synchronized with an R-wave and/or pertaining to the treatment of tachycardia. Defibrillation shocks are generally of moderate to high energy level (i.e., corresponding to thresholds in the range of approximately 5 J to approximately 40 J), delivered asynchronously (since R-waves may be too disorganized), and pertaining exclusively to the treatment of fibrillation. Accordingly, the microcontroller 220 is capable of controlling the synchronous or asynchronous delivery of the shocking pulses.

### 5. ICD Programmer

As indicated above, the operating parameters of ICD 100 may be non-invasively programmed into the memory 260 through a telemetry circuit 264 in telemetric communication via communication link 266 with the external device 254. The external device 254 may be a general purpose computer running custom software for programming ICD 100, a dedicated external programmer device of ICD 100, a transtelephonic transceiver, or a diagnostic system analyzer. Generically, all such devices may be understood as embodying computers, computational devices, or computational systems with supporting hardware or software which enable interaction with, data reception from, and programming of ICD 100.

Throughout this document, where a person is intended to program or monitor ICD 100 (where such person is typically a physician or other medical professional or clinician), the person is always referred to as a “human programmer” or as a “user”. The term “human programmer” may be viewed as synonymous with “a person who is a user of an ICD programming device”, or simply with a “user”. Any other reference to “programmer” or similar terms, such as “ICD programmer”, “external programmer”, “programming device”, etc., refers specifically to the hardware, firmware, software, and/or physical communications links used to interface with and program ICD 100.

The terms “computer program”, “computer code”, and “computer control logic” are generally used synonymously and interchangeably in this document to refer to the instructions or code which control the behavior of a computational system. The term “software” may be employed as well, it being understood however that the associated code may in some embodiments be implemented via firmware or hardware, rather than as software in the strict sense of the term (e.g., as computer code stored on a removable medium, or transferred via a network connection, etc.).

A “computer program product” or “computational system program product” is a medium (for example, a magnetic disk drive, magnetic tape, optical disk (e.g., CD, DVD), firmware, ROM, PROM, flash memory, a network connection to a server from which software may be downloaded, etc) which is suitable for use in a computer or computation system, or suitable for input into a computer or computational system, where the medium has control logic stored therein for causing a processor of the computational system to execute computer code or a computer program. Such medium, also referred to as “computer program medium”, “computer usable medium”, and “computational system usable medium”, are discussed further below.

FIG. 3 presents a system diagram representing an exemplary computer, computational system, or other programming device, which will be referred to for convenience as ICD programmer 254. It will be understood that while the device is referred to an “ICD programmer”, indicating that the device may send programming data, programming instructions, programming code, and/or programming parameters to ICD 100, the ICD programmer 254 may receive data from ICD 100 as well, and may display the received data in a variety of formats, analyze the received data, store the received data in a variety of formats, transmit the received data to other computer systems or technologies, and perform other tasks related to operational and/or physiologic data received from ICD 100.

ICD programmer 254 may include one or more processors, such as processor 304. Processor 304 is used for standard computational tasks well known in the art, such as retrieving instructions from a memory, processing the instructions, receiving data from memory, performing calculations and analyses on the data in accordance with the previously indicated instructions, storing the results of calculations back to memory, programming other internal devices within ICD programmer 254, and transmitting data to and receiving data from various external devices such as ICD 100. Any of the above computational tasks may alternately be performed in a remotely located processor, not shown, that also receives and stores data gathered by the components of ICD 100.

Processor 304 is connected to a communication infrastructure 306 which is typically an internal communications bus of ICD programmer 254; however, if ICD programmer 254 is implemented in whole or in part as a distributed system, communication infrastructure 306 may further include or may be a network connection.

ICD programmer 254 may include a display interface 302 that forwards graphics, text, and other data from the communication infrastructure 306 (or from a frame buffer not shown) for display on a display unit 330. The display unit may be, for example, a CRT, an LCD, or some other display device. Display unit 330 may also be more generally understood as any device which may convey data to a human programmer.

Display unit 330 may also be used to present a user interface which displays internal features of, operating modes or parameters of, or data from ICD 100. The user interface presented via display unit 330 of ICD programmer 254 may include various options that may be selected, deselected, or otherwise changed or modified by a human programmer of ICD 100. The options for programming the ICD 100 may be presented to the human programmer via the user interface in the form of buttons, check boxes, menu options, dialog boxes, text entry fields, or other icons or means of visual display well known in the art.

ICD programmer 254 may include a data entry interface 342 that accepts data entry from a human programmer via data entry devices 340. Such data entry devices 340 may include, for example and without limitation, a keyboard, a mouse, a touchpad, a touch-sensitive screen, a microphone for voice input, or other means of data entry, which the human programmer uses in conjunction with display unit 330 in a manner well known in the art. For example, either a mouse or keystrokes entered on a keyboard may be used to select check boxes, option buttons, menu items, or other display elements indicating human programmer choices for programming ICD 100. Direct text entry may be employed as well. Data entry device 340 may also take other forms, such as a dedicated control panel with specialized buttons and/or other mechanical elements or tactile sensitive elements for programming ICD 100.

In the context of the present system and method, display interface 302 may present on display unit 330 a variety of data related to patient cardiac function and performance, and also data related to the current operating mode, operational state, or operating parameters of ICD 100. Modifications to ICD 100 operational state(s) may be accepted via data entry interface 342 and data entry device 340. In general, any interface means which enables a human programmer to interact with and program ICD 100 may be employed. In one embodiment, for example, a visual data display may be combined with tactile data entry via a touch-screen display.

In another embodiment, a system of auditory output (such as a speaker or headset and suitable output port for same, not shown) may be employed to output data relayed from ICD 100, and a system of verbal input (such as a microphone and suitable microphone port, not shown) may be employed to program ICD 100. Other modes of input and output means may be employed as well including, for example and without limitation, a remote interaction with ICD 100, viewing printed data which has been downloaded from ICD 100, or the programming of ICD 100 via a previously coded program script.

All such means of receiving data from ICD 100 and/or programming ICD 100 constitute an interface 302, 330, 342, 340 between ICD 100 and a human programmer of ICD 100, where the interface is enabled via both the input/output hardware (e.g., display screen, mouse, keyboard, touchscreen, speakers, microphone, input/output ports, etc.) and the hardware, firmware, and/or software of ICD programmer 254.

ICD programmer 254 also includes a main memory 308, preferably random access memory (RAM), and may also include a secondary memory 310. The secondary memory 310 may include, for example, a hard disk drive 312 and/or a removable storage drive 314, representing a floppy disk drive, a magnetic tape drive, an optical disk drive, etc. The removable storage drive 314 reads from and/or writes to a removable storage unit 318 in a well known manner. Removable storage unit 318 represents a magnetic disk, magnetic tape, optical disk, etc. which is read by and written to by removable storage drive 314. As will be appreciated, the removable storage unit 318 includes a computer usable storage medium having stored therein computer software and/or data.

In alternative embodiments, secondary memory 310 may include other similar devices for allowing computer programs or other instructions to be loaded into ICD programmer 254. Such devices may include, for example, a removable storage unit 322 and an interface 320. Examples of such may include a program cartridge and cartridge interface (such as that found in video game devices), a removable memory chip (such as an erasable programmable read only memory (EPROM), programmable read only memory (PROM), or flash memory) and associated socket, and other removable storage units 322 and interfaces 320, which allow software and data to be transferred from the removable storage unit 322 to ICD programmer 254.

ICD programmer 254 also contains a communications link 266 to ICD 100, which may be comprised in part of a dedicated port of ICD programmer 254. From the perspective of ICD programmer 254, communications link 266 may also be viewed as an ICD interface. Communications link 266 enables two-way communications of data between ICD programmer 254 and ICD 100. Communications link 266 has been discussed above (see the discussion of FIG. 2).

ICD programmer 254 may also include a communications interface 324. Communications interface 324 allows software and data to be transferred between ICD programmer 254 and other external devices (apart from ICD 100). Examples of communications interface 324 may include a modem, a network interface (such as an Ethernet card), a communications port, a Personal Computer Memory Card International Association (PCMCIA) slot and card, a USB port, an IEEE 1339 (FireWire port), an infrared port, etc. Software and data transferred via communications interface 324 are in the form of signals 328 which may be electronic, electromagnetic, optical or other signals capable of being received by communications interface 324. These signals 328 are provided to communications interface 324 via a communications path (e.g., channel) 326. This channel 326 carries signals 328 and may be implemented using wire or cable, fiber optics, a telephone line, a cellular link, an radio frequency (RF) link, an infrared link, a microwave link, and other communications channels.

The terms “computer program medium”, “computer usable medium”, and “computational system usable medium” are used, synonymously, to generally refer to media such as removable storage drive 314 and/or removable storage unit 381, a hard disk installed in hard disk drive 312, or secondary memory interface 320 and/or removable storage unit 322. These computer program products or computational system program products provide software to ICD programmer 254.

It should be noted, however, that it is not necessarily the case that the necessary software, computer code, or computer program (any of which may also referred to as computer control logic) be loaded into ICD programmer 254 via a removable storage medium. Such computer program may be loaded into ICD programmer 254 via communications link 328, or may be stored in memory 308 of ICD programmer 254. Computer programs are stored in main memory 308 and/or secondary memory 310. Computer programs may also be received via communications interface 324. Such computer programs, when executed, enable the ICD programmer 254 to perform the features of the present system and method, as discussed herein.

In particular, the computer programs, when executed, enable the processor 304 to perform several features of the present system and method. Accordingly, such computer programs represent controllers of ICD programmer 254, and thereby controllers of ICD 100.

In one embodiment of the system and method described herein, all pertinent instructions and data are stored or implemented entirely in ICD programmer 254. In an alternative embodiment of the system and method described herein, some pertinent instructions and data are stored or implemented in-part within ICD 100 and in-part within ICD programmer 254. Based on the discussion below, it will be apparent to persons skilled in the relevant art how the requisite operations and data may be distributed, in a variety of ways, between ICD 100 and ICD programmer 254.

### 6. The System and Method at Hand

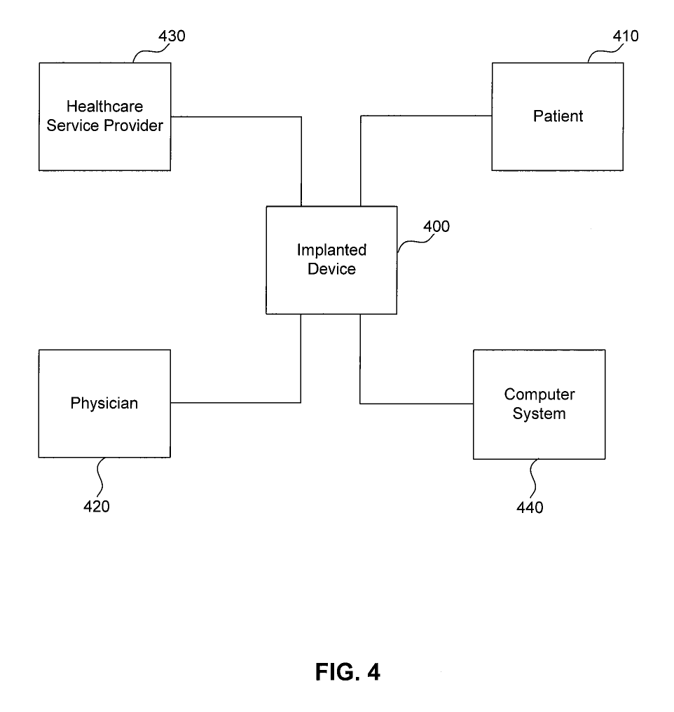

FIG. 4 is a high-level block diagram of a method of detecting irregularities in a patient's metabolic function. To begin with, an implantable device 400, such as the ICD 100 described above, is implanted into a patient. Implantable device 400 collects physiological data from the patient, and thereby serves as the center of information distribution. Implantable device 400 may automatically distribute the physiological data it collects, or may receive a command to upload the physiological data. The physiological data may be delivered to any one or more parties or system components, such as patient 410, physician 420, healthcare service provider 430, and/or computer system 440. The patient 410, physician 420, healthcare service provider 430, and/or computer system 440 may then act on the received physiological data. The physiological data may be used to detect irregular metabolic function and develop a treatment plan to resolve the irregularity or treat the symptoms caused by the irregularity.

For example, when a patient has suffered from HF, the patient is usually treated by having a pacemaker or ICD implanted to manage the pacing and healthy functioning of the heart. In addition to the implantation of the device, the patient is usually prescribed various medications to control things such as their blood pressure, cholesterol, coagulation, etc. The patient may also take other medication for conditions unrelated to the HF that could also affect metabolism. Medications that can interfere with normal metabolic function include lithium carbonate, amiodarone (Amiodarone, Cordarone, or Pacerone), and inteferon alfa (Infergen, Rebetron, and Wellferon). As would be understood by one of skill in the art, this list is non-exclusive and is meant only to present examples of medications that can affect metabolic function.

In addition, the HF may also induce a lifestyle change which causes the patient to change their activity level. For example, the patient may have to cease participation in active sports for some period of time after the HF. Over time, the patient may have periods in which their activity level varies. These changes in activity level can also alter a patient's metabolism. Thus, it can be difficult to determine if changes in a patient's metabolism stem from a metabolic disease, such as a thyroid-related disease, or a reaction to prescribed medication, or the result of a change in activity level.

If the metabolic change is caused by a change in activity level, and the metabolic rate remains within acceptable parameters, corrective action may not be required. However, a relatively small change in metabolic rate that is not accompanied by a corresponding change in activity level may indicate onset of a serious metabolic irregularity or a reaction to a medication. Because symptoms of a metabolic disease can develop gradually and can be difficult for a patient or physician to recognize until the disease has progressed, identifying the underlying cause of even a small change in metabolism can be crucial to a patient's health.

The method of FIG. 4 provides an implantable device 400 which is implanted into the patient and provides a continuous measure of physiological parameters indicative of metabolic function. For example, device 400 can measure changes in the patient's metabolic rate when compared with the patient's activity level, as determined by a motion sensor or a respiratory rate sensor. This information can be provided to the physician, who can then more accurately determine if the change in metabolism could be indicative of a metabolic disease or a reaction to medication that requires management.

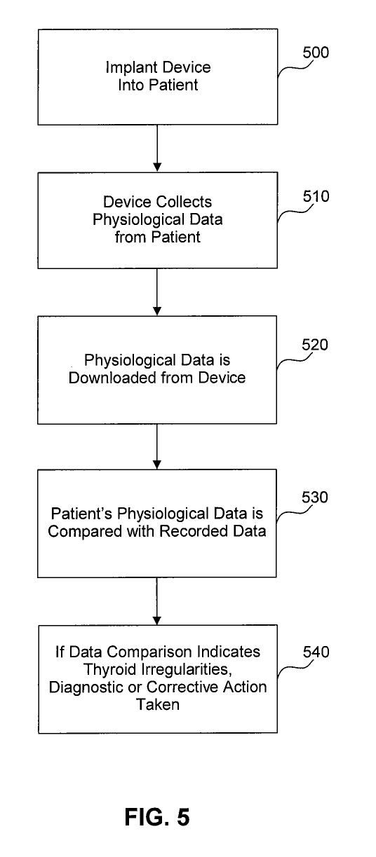

FIG. 5 is a flow chart showing a method for detecting and treating irregular metabolic function. In step 500, a device is implanted into a patient. For example, an implantable device, such as ICD 100, described above, may be implanted into a patient who has suffered from HF. The implantable device includes the appropriate circuitry, and at least one physiological sensor which collects data such as the patient's activity level, respiratory rate, temperature, blood pressure, cardiac output, and/or oxygen saturation. In step 510, the implanted device collects the physiological data from the patient. In step 520, the collected physiological data is downloaded from the implanted device. Alternately, the physiological data can be recorded and stored by the device itself. In step 530, the patient's physiological data is compared with archived data for that particular patient. For example, if the physiological data includes the patient's activity level and also provides sufficient information from which to calculate the patient's metabolic rate, then the data may show that the patient has experienced an abnormal drop or rise in metabolic rate when compared with previous measurements taken from the same patient at a similar activity level. Thus, in step 540, if the patient's metabolic rate indicates possible irregularities in metaboilc function, additional diagnostic and/or corrective action can be taken.

The physiological data collected in step 510 may be directly analyzed by the implanted device, or may be analyzed by an external system, such as computer system 440 in FIG. 4. Similarly, the collected physiological data can be stored directly by the implanted device or an external system, or both. For example, the physiological data may be patient's activity level and respiratory rate over time. The device or computer system may then apply appropriate algorithms to compare the patient's current metabolic rate and current activity level to historical records of that patient's metabolic rate and respective activity level. Similarly, if the physiological data is the patient's respiratory level over time, as collected from a respiratory sensor, the device or computer system may apply appropriate algorithms to the data to determine if the metabolic rate of the patient compared to the patients current respiratory level is within threshold levels when compared with historical records of that patient's metabolic rate at certain respiratory levels. The algorithm may take into account both metabolic rate and respiratory rate to further increase the accuracy of the comparison.

For ease of comparison between the patient's current condition and archived patient data, the device may calculate a single index number based on the patient's current activity level and current metabolic rate. This index number may then be easily compared with archived indexes, either by the device itself, an external processor, or a healthcare service provider, to determine whether the patient's metabolic rate, taking into account the patient's activity level and/or respiratory rate, is higher or lower than would be expected based on the patient's historical data. The index number may be transmitted to a healthcare service provider as an indicator of possible metabolic irregularity.

The collected physiological data may include the patient's oxygen consumption. The patient's oxygen consumption (V0₂) may be computed, and the patient's metabolic rate approximated, by using the Fick equation. The Fick equation provides a means of measuring oxygen consumption by a calculation involving arterial oxygen saturation (SaO₂) (or assuming it to be constant at 99%), mixed venous oxygen saturation (SvO₂), and cardiac output (CO). Cardiac output may be measured using reflectance oximetry. Alternatively, cardiac output may be measured using impedance cardiography, or any other technique known in the art to measure cardiac output. SaO₂ may be measured by an implantable pulse oximeter embedded in ICD 100. SvO₂ may be measured by a sensor placed in the right ventricle, right atrium, or pulmonary arteries. According to the Fick equation, V0₂ is calculated as follows:

V0₂=CO(SaO₂-SvO₂).1.34.Hgb.1000

The units for CO are in liters per minute, Hgb is in grams of hemoglobin per 1000 ml of blood, SaO₂ and SvO₂ are in fractions.

In a simplification of this equation, the cardiac output may be assumed to be related to the mixed venous oxygen saturation. This relation may be initially estimated by assuming that the cardiac output is directly related to the mixed venous saturation using a linear relationship. Alternatively, the cardiac output may also be directly related to the activity level or the intrinsic heart rate. Cardiac output may also be estimated by some other means including Doppler or thermal dilution measurements.

Various alternative systems and methods of obtaining cardiac output are known to one of skill in the art. For example, systems and methods of obtaining cardiac output are described in U.S. Pat. Nos. 5,788,647; 6,306,098; 7,139,609; and 7,164,948. The systems and methods described in these patents, as well as other known systems and methods, may be used in conjunction with the systems and methods described herein, and are therefore incorporated in their entirety by reference.

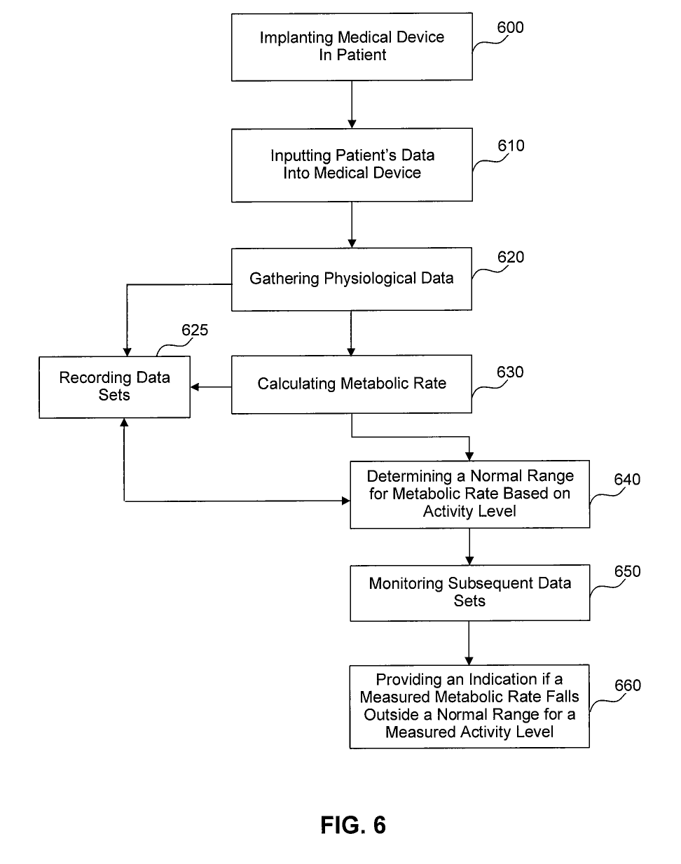

FIG. 6 is a flow chart showing an alternative method for detecting irregular metabolic function in a patient. In step 600, a device is implanted into a patient. In step 610, patient data is inputted into the device. In alternative embodiments, step 610 is performed before step 600, or may not be performed. If patient data is inputted, the data may include information such as the patient's height, weight, waist dimensions, and/or somatotype (endomorphic, ectomorphic, or mesomorphic). The patient data may also include historical data relating to the patient's metabolic rate, activity level, and respiratory level. In step 620, which occurs on an ongoing basis, the device gathers physiological data. As physiological data is gathered, the device records the data as data sets (step 625), and may also transmit the data to a separate location for storage. Each data set includes at least two physiological parameters measured or calculated at any given time. For example, each data set may include data indicative of the patient's metabolic rate, respiratory rate, and/or activity level, in addition to any other physiological parameter. Based on the collected and/or historical data, the patient's current metabolic rate is calculated in step 630. In step 640, the device determines a normal range for the patients metabolic rate based on activity level as recorded in the data sets. In an alternative embodiment, the normal range could be determined by reference to patient data collected prior to implantation of the medical device. Although steps 640 and 660 in FIG. 6 are directed to metabolic rate as it relates to the patient's activity level, various different physiological parameters can be compared with metabolic rate to achieve the goals of the method. For example, respiratory rate can substitute for metabolic rate in steps 640 and 660. In an alternative embodiment, both activity level and metabolic rate are considered in relation to the patient's metabolic rate.

In step 650, the device monitors subsequent data sets, comparing the physiological parameters in the data sets to the determined normal range. In step 660, the device provides an indication if a measured metabolic rate falls outside the determined normal range for a measured activity level. As described above, additional physiological parameters can substitute for activity level. For example, respiratory rate can substitute for activity level, or both respiratory rate and activity level may be considered. It should be understood that even if a specific measured activity level has no precedent in a previous data set, the implantable medical device can determine the appropriate normal range for metabolic rate at that activity level by extrapolating the data contained in recorded data sets.

In this way, the device monitors for changes in metabolic rate that are attributable solely to a change in activity level. As such, the device can be used to more accurately identify onset of metabolic diseases as well as changes in metabolism caused by medications. The device allows for early detection of changes in metabolism.

The information obtained and calculated by the implanted device may be downloaded by a healthcare service provider and/or computer system, and may be used to determine if a patient is experiencing irregular metabolic function. The remotely stored information may be updated monthly, weekly, or even daily. For example, the device may perform all calculations and comparisons internally and remotely alert a healthcare service provider when it is determined that a patient may be experiencing irregular metabolic function. Alternately, the device may alert the patient directly by an alerting noise, vibration, etc.

### EXAMPLE 1

Provided is a method for detecting irregularities in metabolic function. The method includes the steps of: i) implanting a medical device into a patient, wherein the medical device measures one or more physiological parameters indicative of metabolic rate and activity level; ii) recording data sets including a patient's metabolic rate and activity level, wherein each activity level measurement is associated with a corresponding metabolic rate measurement; iii) determining a normal range for metabolic rate based on activity level; iv) monitoring subsequent data sets; v) providing an indication if metabolic rate in a subsequent data sets falls outside of the normal range for a measured activity level; and vii) preparing a treatment program to address the cause of the irregular metabolic function. As would be apparent to one of skill in the art, the method presented does not have to be performed in the exact sequential order listed above. Instead, the method may be performed in any logical order deemed fit by one of skill in the art.

In practice, the medical device includes a motion sensor adapted to measure relative movement by the patient. For example, the motion sensor may be an accelerometer. The medical device can also include a telemetry circuit adapted to deliver physiological data to an external receiver. The physiological data is generally representative of the measured relative movement. The physiological data may be raw and/or analyzed data. For example, the medical device may include a respiratory sensor that is used to calculate the patient's respiratory rate. The patient's respiratory rate may be considered when determining whether any change in metabolic rate is due to an increase in the patient's activity level to more accurately diagnosis possible metabolic irregularity. The medical device may also calculate the patient's metabolic rate, and as such the delivered physiological data may include the patient's metabolic rate.

The treatment program may be prepared once the underlying causes of the metabolic irregularity are determined. For example, if tests confirm that the patient is suffering from hypothyroidism, the doctor may prescribe one of several available thyroid hormones to stimulate hormone production (e.g., levothyroxine sodium). If it is determined that the patient is suffering from hyperthyroidism, treatment may include radioactive iodine therapy or surgical removal of all or a portion of the thyroid gland. In addition, treatment can include neural stimulation, for example, stimulation of the pituitary gland or the hypothalamus, to pace the autonomic nervous system or to control the release of hormones. This stimulation can be achieved by a lead positioned in the area of the brain where stimulation is desired. If the metabolic irregularity is cause by medications the patient is taking, the dosage of those medicines can be reduced or replacement medicines can be used.

The steps of recording data sets and determining a normal range for metabolic rate based on activity level may be performed by a healthcare service provider, a processor within the medical device, or an external receiver. The amount of acceptable variance between the archived condition data and data regarding the patient's current condition can be determined by a programmer or by a pre-programmed algorithm. Once the appropriate range is determined, the programmer can set the device or external system to alert the patient or healthcare provider when the normal range is exceeded, which could indicate a metabolic irregularity. The patient may then consult with the healthcare provider to determine whether corrective steps are required. If the patient is taking medications that have been known to effect metabolic function, the healthcare provider may instruct the patient to cease taking the medication until the cause of the metabolic irregularity can be confirmed. Alternately, the healthcare provider can prescribe an alternate medication. No matter the underlying cause of the metabolic irregularity, the early detection provided by the monitoring device allows for an increased chance of treating the condition before symptoms become more serious.

### 7. Conclusion

It is to be appreciated that the Detailed Description section, and not the Summary and Abstract sections, is intended to be used to interpret the claims. The Summary and Abstract sections may set forth one or more but not all exemplary embodiments of the present system and method as contemplated by the inventors, and thus, are not intended to limit the present method and system and the appended claims in any way.

Moreover, while various embodiments of the present system and method have been described above, it should be understood that they have been presented by way of example, and not limitation. It will be apparent to persons skilled in the relevant arts that various changes in form and detail can be made therein without departing from the spirit and scope of the present system and method. Thus, the present system and method should not be limited by any of the above described exemplary embodiments, but should be defined only in accordance with the following claims and their equivalents.

In addition, it should be understood that the figures and screen shots illustrated in the attachments, which highlight the functionality and advantages of the present system and method, are presented for example purposes only. The architecture of the present system and method is sufficiently flexible and configurable, such that it may be utilized (and navigated) in ways other than that shown in the accompanying figures. Moreover, the steps indicated in the exemplary systems and methods described above may in some cases be performed in a different order than the order described, and some steps may be added, modified, or removed, without departing from the spirit and scope of the present system and method.

Further, the purpose of the foregoing Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the present system and method in any way.

What is claimed is:

1. A method for detecting irregularities in metabolic function, comprising:
implanting a medical device into a patient, wherein the medical device measures one or more physiological parameters indicative of metabolic rate and activity level;
collecting data sets, wherein each data set includes a patient's metabolic rate and corresponding activity level;
calculating a normal range for metabolic rate based on corresponding activity level;
monitoring subsequent data sets;
providing an indication if metabolic rate in a subsequent data set falls outside of the normal range for a corresponding activity level.

2. The method of claim 1, wherein said indication is an audible sound that notifies the patient.

3. The method of claim 1, wherein said indication is a vibration that notifies the patient.

4. The method of claim 1, wherein said indication notifies a remote healthcare provider.

5. The method of claim 1, wherein the physiological parameter indicative of metabolic rate includes a measure of the patient's mixed venous saturation, cardiac output, and arterial oxygen saturation.

6. The method of claim 5, wherein the metabolic rate is calculated using a Fick equation.

7. The method of claim 1, further comprising:
performing diagnostic testing on the patient to determine if the patient is experiencing irregular metabolic function if the patient's metabolic rate falls outside of the normal range for a measured activity level.

8. The method of claim 7, further comprising:
preparing a treatment plan if the diagnostic testing indicates that the patient is experiencing irregular metabolic function.

9. The method of claim 8, further comprising:
adjusting the patient's medication to resolve the irregular metabolic function.

10. The method of claim 8, further comprising:
prescribing a thyroid hormone to treat the irregular metabolic function.

11. The method of claim 8, further comprising:
performing radioactive iodine therapy on the patient to treat the irregular metabolic function.

12. The method of claim 8, further comprising:
positioning a lead in an area of the patient's brain to pace the patient's autonomic nervous system.

13. The method of claim 8, further comprising:
positioning a lead in an area of the patient's brain to control release of hormones.

14. The method of claim 1, further comprising:
measuring data indicative of the patient's respiratory rate.

15. The method of claim 14, wherein the step of calculating a normal range for metabolic rate further comprises determining whether a change in respiratory rate is due to a change in the patient's activity level.

16. The method of claim 1, wherein the step of calculating a normal range is conducted by a processor within the medical device.

17. The method of claim 1, wherein the step of calculating a normal range is conducted by an external receiver.

18. A implantable medical device that detects irregularities in metabolic function, comprising:
one or more sensors adapted to measure one or more physiological parameters indicative of metabolic rate and activity level;
circuitry adapted to collect data sets, wherein each data set includes a patient's metabolic rate and corresponding activity level;
circuitry adapted to calculate a normal range for metabolic rate based on corresponding activity level;
circuitry adapted to monitor subsequent data sets and to provide an indication if a metabolic rate in a subsequent data set falls outside of the normal range for a corresponding activity level.

19. The implantable medical device of claim 18, wherein the physiological parameter indicative of metabolic rate comprises a measure of the patient's mixed venous saturation, cardiac output, and arterial oxygen saturation.
